(19) United States
(12) Patent Application Publication
(10) Pub. No.: US 2025/0218564 A1
(43) Pub. Date: Jul. 3, 2025

(54) COMPUTER VISION-BASED NUTRITION TRACKING

(71) Applicant: TOSHIBA GLOBAL COMMERCE SOLUTIONS, INC., Durham, NC (US)

(72) Inventors: David J. STEINER, Durham, NC (US); Craig COMPTON, Wake Forest, NC (US); Daniel J. HUNT, Wake Forest, NC (US)

(21) Appl. No.: 18/401,179

(22) Filed: Dec. 29, 2023

(51) Int. Cl. G16H 20/60; G03B 21/00; G06V 10/82; G06V 20/68; G08B 21/18

(52) U.S. Cl. CPC G16H 20/60; G03B 21/00; G06V 10/82; G06V 20/68; G08B 21/182

(57) ABSTRACT

Method and apparatus for computer vision-based tracking are provided. A set of images depicting a set of items in a receptacle of a user is accessed. At least a first item, of the set of items, is identified based on processing at least a first image of the set of images using one or more object recognition machine learning models. Based on a mapping, a caloric value of the first item is determined, and nutrition tracking information for the user is updated based on the caloric value. A set of user characteristics provided by the user is determined. In response to determining that the updated nutrition tracking information satisfies one or more criteria based on the set of user characteristics, a notification is transmitted to a mobile device of the user.

### BACKGROUND

A variety of machine learning model architectures have been introduced in recent years to perform a diverse assortment of tasks. For example, in the computer vision space, machine learning has been used to perform image segmentation, object detection, depth estimation, and the like. Many conventional computer vision models impose substantial computational costs during training and use, often consuming considerable hardware resources and introducing significant latency before a prediction can be returned. Additionally, conventional computer vision systems are generally quite limited in terms of their flexibility and adaptability to new tasks.

### BRIEF DESCRIPTION OF THE DRAWINGS

FIG. 1 depicts an example environment for computer vision-based nutrition tracking, according to some embodiments of the present disclosure.

FIG. 2 depicts an example augmented receptacle with computer vision-based visualizations for a specific item, according to some embodiments of the present disclosure.

FIG. 3 depicts an example augmented receptacle with computer vision-based visualizations for a set of items, according to some embodiments of the present disclosure.

FIG. 4 is a flow diagram depicting an example method for generating computer vision-based notifications, according to some embodiments of the present disclosure.

FIG. 5 is a flow diagram depicting an example method for outputting computer vision-based notifications, according to some embodiments of the present disclosure.

FIG. 6 is a flow diagram depicting an example method for generating notifications, according to some embodiments of the present disclosure.

FIG. 7 depicts an example computing device for computer vision-based nutrition tracking, according to some embodiments of the present disclosure.

### DETAILED DESCRIPTION

Embodiments of the present disclosure provide techniques and systems for improved computer vision-based evaluation and prediction of future nutrition, as well as generation of dynamic notifications to improve selection and use of sustenance alternatives in a seamless and efficient manner.

### Advantages of Computer-Vision Tracking

In a wide variety of environments, tracking and identifying physical objects or items is a complex and difficult task. In some embodiments, one or more machine learning-based models (e.g., convolutional neural networks (CNNs)) may be used to identify objects depicted in captured images. In many realistic settings, objects may be partially or entirely obscured in the captured image(s). In some embodiments of the present disclosure, by utilizing multiple imaging sensors and/or capturing images from multiple angles and/or at multiple times, a more accurate understanding of the relevant objects can be obtained.

Additionally, in many settings, the nutritive contents of consumable items (e.g., caloric value, vitamin content, and the like) are difficult to determine, parse, and understand. This is particularly true when items may be consumed or shared by multiple users, portion(s) of the items may be discarded without being consumed, items are consumed over a relatively long period of time, and the like. In some embodiments, a wide variety of contextual information can be collected and evaluated to provide more accurate and reliable nutrition predictions, as compared to conventional approaches. In some aspects, such more accurate predictions can substantially improve the functionality of the tracking systems, such as by enabling improved output for downstream processes.

In some embodiments, the computer-vision and contextual tracking described herein can reduce computational expense of the tracking systems. For example, by evaluating various contextual information, the system may generate accurate predictions using fewer resources (e.g., processing a single set of image(s) once for a given user). In contrast, some conventional approaches rely on repeated evaluations (e.g., processing images using a machine learning model multiple times, such as daily, before each meal, and the like). As such computer vision models are often resource-intensive (e.g., requiring substantial memory, processor time, and/or power consumption to evaluate images), such repeated analysis consumes substantial computational resources and energy. In contrast, embodiments of the present disclosure enable a one-time analysis (e.g., evaluating images only when the items are selected, rather than repeatedly each day) based on additional user context that improves accuracy without the need for additional processing. In these ways, aspects of the present disclosure provide improved machine learning predictions with reduced computational expense.

### Example Environment for Computer Vision-Based Nutrition Tracking

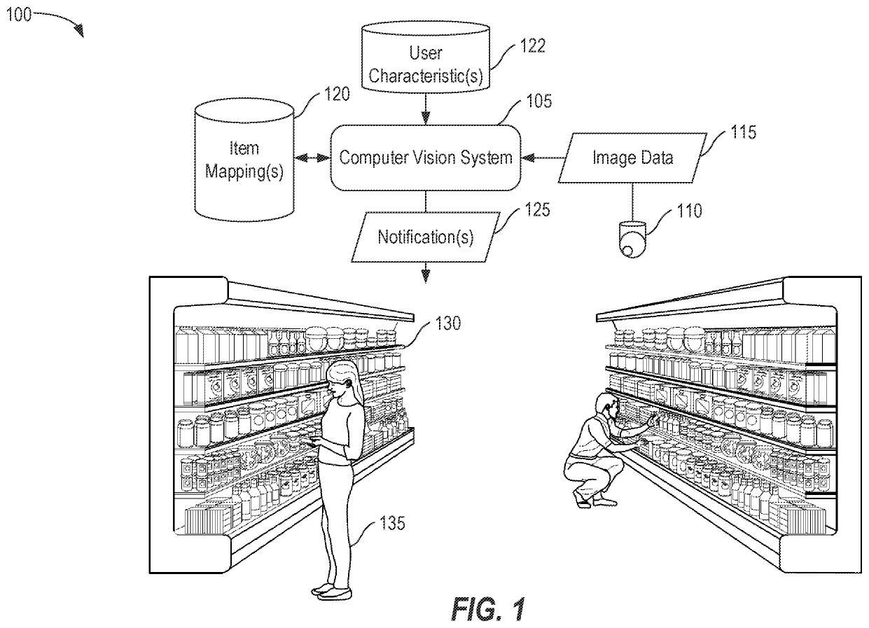

FIG. 1 depicts an example environment 100 for computer vision-based nutrition tracking, according to some embodiments of the present disclosure.

The illustrated environment 100 depicts a physical space or location (e.g., a retail establishment) where users 135 can select or obtain items 130. For example, the environment 100 may correspond to a grocery store, and the items 130 may correspond to or comprise consumable objects (e.g., food and/or drinks). In the illustrated embodiment, the environment 100 includes one or more imaging sensors 110 (e.g., cameras). Though a single imaging sensor 110 is depicted for conceptual clarity, in some aspects, there may be multiple such sensors arranged in the space. For example, there may be one or more imaging sensors 110 mounted on a ceiling of the space, one or more imaging sensors 110 mounted on or near the shelves that hold items 130 in the space, one or more imaging sensors 110 located at or near a check-out or exit of the space, one or more imaging sensors 110 located in the receptacles (e.g., carts and/or baskets) used by users 135, and the like.

Additionally, although the illustrated example depicts imaging sensors 110, in some embodiments the environment 100 may additionally or alternatively include other sensors, such as pressure sensors, weight sensors, audio sensors (e.g., microphones), radar and/or LIDAR sensors, and the like. Generally, the sensors (including imaging sensors 110) may be used to collect data that is used to identify which item(s) 130 have been selected by user(s) 135 in the space.

In the illustrated example, the imaging sensors 110 generate image data 115 (e.g., individual images or frames, or a sequence of frames or images, such as a video file). The image data 115 is accessed by a computer vision system 105 for processing. As used herein, “accessing” data may generally include receiving, requesting, retrieving, generating, collecting, obtaining, or otherwise gaining access to the data from one or more local and/or remote sources. In the illustrated example, the computer vision system 105 evaluates the image data 115 and a set of item mapping(s) 120 to generate notifications 125 for users 135.

In some embodiments, the computer vision system 105 uses one or more machine learning models (e.g., CNNs) to identify items 130 depicted in the image data 115. For example, the computer vision system 105 may process image(s) depicting a receptacle of a given user 135 in order to identify which item(s) 130 the user 135 has selected and placed in their receptacle. In some embodiments, the machine learning models comprise or correspond to computer vision models which can be used to perform tasks such as object detection and/or recognition, text recognition, image segmentation, and the like.

In some embodiments, the computer vision system 105 generates a set of items indicating the item(s) 130 that are depicted in the image data 115 with respect to a given user 135. For example, a user 135 may opt-in to the nutrition tracking system (e.g., using a smartphone application or via a loyalty program). In response, when the user 135 is detected in the environment 100, the computer vision system 105 may capture and evaluate image data 115 to generate a list of the item(s) 130 that the user 135 has selected. Generally, the user 135 may be identified using a variety of techniques, such as facial recognition, detecting a device associated with the user 135 (e.g., determining that the user's smartphone has connected to the wireless network in the environment 100), and the like.

In some embodiments, the image data 115 can be captured and/or evaluated while the user 135 traverses the environment 100. For example, each time the user 135 places a new item 130 into their receptacle, the computer vision system 105 may generate an updated list of items for the user. In some embodiments, the image data 115 may be captured and/or evaluated while the user checks out or otherwise prepares to exit the environment.

In the illustrated example, the computer vision system 105 accesses and/or evaluates a set of item mappings 120 based on the detected set of items 130 in the user's receptacle. Generally, the item mappings 120 indicate nutrition information for items 130 that can be selected in the environment 100. For example, the item mappings 120 may indicate the caloric value(s) of the item(s). Generally, the caloric values may indicate the number of nutrition calories in the item, in each serving of the item, and the like. For example, different brands and/or types of food (e.g., different brands of pretzel) may have differing caloric values. By specifically identifying each item (e.g., using object detection and/or image or text recognition to determine the specific brand and item size), the computer vision system 105 may be able to access accurate caloric information based on the item mappings 120. Although caloric tracking is used herein as one example use case, aspects of the present disclosure can be readily used to monitor or evaluate consumption a wide variety of nutrition elements, such as salt, cholesterol, sugar, dietary fiber, and the like.

In some aspects, the computer vision system 105 may access item mappings 120 each time a new item is detected for the user. In some embodiments, based on the item mappings 120, the computer vision system 105 can generate and/or update nutrition tracking information for the user based on the determined caloric values. For example, the computer vision system 105 may determine a total number of calories present in the item(s) that the user has selected.

In the illustrated example, the computer vision system 105 may also access or determine a set of user characteristics 122 for the user 135. These user characteristics 122 may be determined from a number of sources. For example, in some embodiments, the user 135 may input or specify the characteristics (e.g., via a smartphone application and/or loyalty program). Generally, the user characteristics 122 may include a variety of information that may be relevant or useful to contextualize the nutrition tracking information for the user.

For example, in some aspects, the user characteristics 122 may indicate information such as the number of people for whom the user 135 is shopping (e.g., the number of people in the user's family and/or the number of people that will share or consume the items 130 being selected), and/or attributes of these related users (e.g., their ages, heights, weights, activity levels, target calorie intake, and the like). In some embodiments, the user characteristics 122 may indicate attributes of the specific user 135, such as their target calorie intake (e.g., specified by the user and/or inferred based on other user attributes), the user's age, height, weight, activity level, and the like. In some embodiments, the user characteristics 122 may indicate the number of meals and/or duration of time over which the items will be consumed. For example, the user may specify that they are shopping for the next seven days, that they plan to prepare four meals using the items 130, and the like.

In some aspects, based on the user characteristics 122 and item mappings 120, the computer vision system 105 can predict the caloric intake for the user 135 (e.g., predicting the number of calories the user will consume per day). For example, by dividing the total calorie count (reflected by the item mappings 120) among the individuals for whom the user 135 is shopping (equally, or in proportion to their attributes, such as in proportion to their target caloric intake and/or age/height/weight/activity level), the computer vision system 105 can predict the number of calories that the user 135 will consume based on the items. Similarly, by dividing this value by the indicated duration (e.g., the number of meals and/or days for which the user is shopping), the computer vision system 105 may determine or predict the caloric intake of the user for each meal, for each day, and the like.

In some aspects, the computer vision system 105 predicts the caloric intake based on additional contextual information, such as a predicted or estimated caloric waste for the items. For example, in addition to the total caloric value of a given item, the item mappings 120 may indicate the estimated or typical caloric waste (e.g., the number of calories that are not consumed). This caloric waste may be due to a variety of causes, such as packaging (e.g., where some portion of the food cannot readily be extracted for consumption), convenience, spoilage (e.g., where the item often expires before being fully consumed), and the like. In some aspects, in addition to or instead of the item mappings 120 indicating predicted waste, the computer vision system 105 may determine estimated waste based on the user characteristics 122. For example, the user 135 may specify or estimate what percentage of food (on a per-item basis or on a general item-agnostic basis) they waste (e.g., throw away without eating). In this way, the caloric intake of the user can be predicted while taking into account the potential waste, resulting in a more accurate estimate.

In some embodiments, the computer vision system 105 can compare the predicted caloric intake of the user 135 with one or more criteria (e.g., target intake for the user) in order to generate notifications 125. In some embodiments, the computer vision system 105 generates notifications 125 selectively based on whether the criteria are satisfied. For example, in some embodiments, the computer vision system 105 refrains from generating a notification 125 if the user's predicted caloric intake is equal to or less than their caloric target.

The notifications 125 may generally take a variety of forms depending on the particular implementation. For example, in some embodiments, the computer vision system 105 may use one or more projection devices to project an indication of the item(s) in the user's receptacle, as discussed in more detail below.

As another example, in some embodiments, the computer vision system 105 may transmit one or more notifications to a user device (e.g., a smartphone) associated with the user. Such notifications may be implemented, for example, using text messaging, email, notification via an application on the mobile device, and the like. In some embodiments, the notification 125 may indicate information such as the predicted caloric intake of the user, the difference between the predicted intake and the user's target intake, and the like. In some embodiments, the notification(s) 125 may indicate which item(s) contribute to the excess. For example, the notification 125 may indicate that the item most-recently added to the receptacle caused the predicted intake to exceed the target. In some embodiments, the computer vision system 105 may indicate which item(s) contribute most to the excess, such as based on the number of calories in each item 130 and/or the calorie density of each item 130.

For example, based on the caloric value and information such as the serving size, mass, and/or volume of the item, the computer vision system 105 can determine which item(s) are most calorie-dense (e.g., which items have high caloric value with low mass and/or volume, and/or high caloric value per serving). In some embodiments, the notification(s) 125 can therefore indicate or rank one or more item(s) 130 in the receptacle based on their calorie density, such as to indicate the top N items with the highest calorie density. By replacing such high-density items, the user may be able to reduce their predicted caloric intake with minimal impact.

In some embodiments, the computer vision system 105 may suggest alternatives for one or more of the items. For example, in response to determining that a specific brand or type of item (e.g., a can of sauce) has a high calorie density (e.g., above a threshold, or in the top N % of items in the receptacle), the computer vision system 105 may identify other substitute items (e.g., other cans of sauce from different brands and/or different types) that have lower calorie value and/or lower calorie density (e.g., based on the item mappings 120). By suggesting such alternatives, the computer vision system 105 can enable the user to readily and easily meet their goals.

Example Augmented Receptacle with Computer Vision-Based Visualizations for a Specific Item

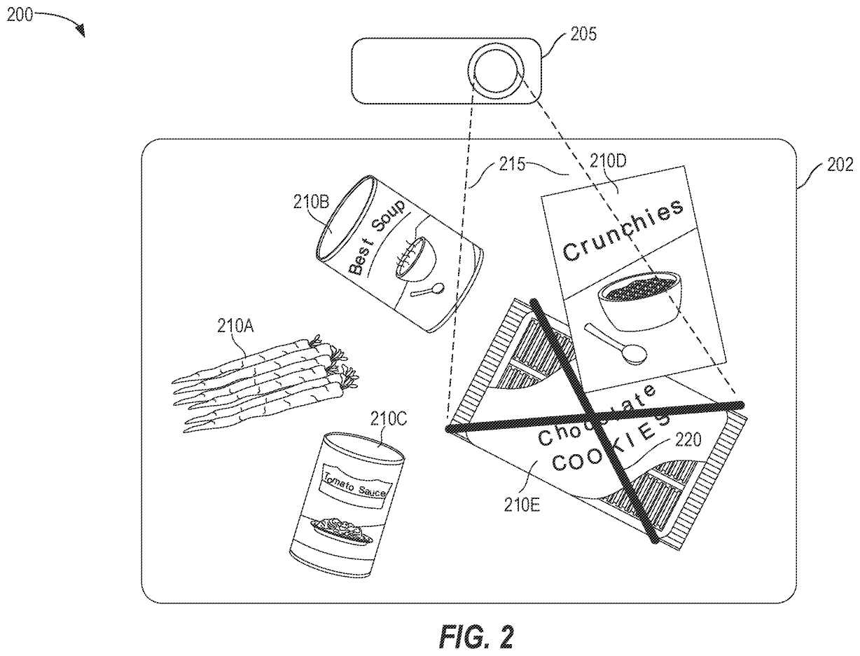

FIG. 2 depicts an example augmented receptacle 200 with computer vision-based visualizations for a specific item, according to some embodiments of the present disclosure.

In the illustrated example, a user receptacle 202 is depicted having a number of items (e.g., items 130 of FIG. 1). The receptacle 202 generally corresponds to any container which may be used, by a user, to contain or carry items in a physical space. For example, the receptacle 202 may correspond to a cart, a basket, a bag, and the like. In some aspects, the receptacle 202 corresponds to the hands of the user (e.g., if the user is carrying items without a basket). As illustrated, there are a number of items 210 in the receptacle 202. For example, the receptacle 202 contains carrots (indicated by item 210A), a can of soup (indicated by item 210B), a can of sauce (indicated by item 210C), a box of cereal (indicated by item 210D), and a container of cookies (indicated by item 210E).

As illustrated, a projector device 205 is projecting a visualization 220 onto the item 210E in the receptacle 202 (as indicated by lines 215). The projector device 205 may generally correspond to or comprise any device capable of projecting visual imagery in an environment. For example, the projector device 205 may be mounted on a ceiling of the environment. Though a single projector device 205 is depicted for conceptual clarity, in some aspects, there may be a number of projectors in the space. For example, projector devices 205 may be installed along each row or aisle, near each camera (e.g., each imaging sensor 110 of FIG. 1), and the like.

In some embodiments, the visualization 220 acts as a notification (e.g., a notification 125 of FIG. 1). For example, the computer vision system may project the visualization 220 in response to determining that the predicted caloric intake of the user meets or exceeds a threshold (e.g., a target value). In the illustrated example, the visualization 220 is a large “X” covering the item 210E. For example, the visualization 220 may be used to indicate that the corresponding item 210E should be removed (or not consumed by the user) to prevent the user from exceeding their target caloric intake.

In some embodiments, to project the visualization 220, the computer vision system may determine, based on the image data depicting the user's receptacle 202 (e.g., image data 115 of FIG. 1), the location(s) of one or more item(s) 210 in the receptacle 202. For example, based on object detection and recognition models, the computer vision system may determine the physical location of each item 210 relative to the cart. Further, based on the predicted caloric information (e.g., calorie density, other nutritional value of each item, and the like), the computer vision system may identify one or more items that should be removed. In the illustrated example, the computer vision system has determined that the cookie item 210E should be removed, and has therefore determined to project the visualization 220 onto the physical location of the item in the receptacle 202, allowing the user to readily-identify the relevant item.

Although depicted as an “X”, the visualization 220 may take a variety of forms depending on the particular implementation. For example, in some aspects, the computer vision system may circle or outline the relevant item(s), project a color overlay onto the relevant item(s), and the like.

In some embodiments, the computer vision system generates the visualization 220 in response to a user request. For example, the user may use an application on their phone to request that the computer vision system identify and indicate which item(s) 210 should be removed in order to reduce the predicted caloric intake. In some embodiments, the computer vision system identifies the items having the highest calorie density. In some embodiments, the computer vision system may also consider other information, such as other nutritional value of the items (e.g., where some items may be highly caloric but also high in other nutrition, while other items may be highly caloric without substantial other nutritional value). In some such embodiments, the computer vision system may indicate item(s) that are high calorie with low other nutritional value (e.g., below a threshold).

In some embodiments, the computer vision system generates the visualization 220 automatically in response to determining that the predicted intake exceeds the user's target caloric intake. For example, suppose the addition of the item 210C to the receptacle 202 causes the predicted caloric intake to exceed the target. In some embodiments, the computer vision system may therefore evaluate each item 210 in the receptacle 202 to determine which item(s) have the highest calorie value and/or calorie density, and/or the lowest nutritional value. These item(s) 210 may then be highlighted using visualizations 220.

Example Augmented Receptacle with Computer Vision-Based Visualizations for a Set of Items

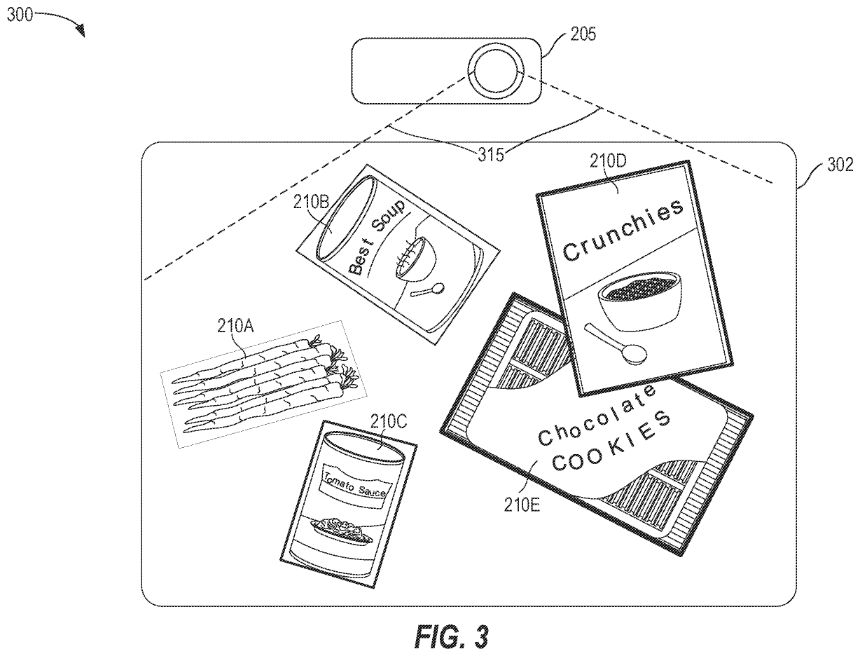

FIG. 3 depicts an example augmented receptacle 300 with computer vision-based visualizations for a set of items, according to some embodiments of the present disclosure.

In the illustrated example, a user receptacle 302 is depicted having a number of items (e.g., items 130 of FIG. 1). As discussed above, the receptacle 302 (which may correspond to the receptacle 202 of FIG. 2) generally corresponds to any container which may be used, by a user, to contain or carry items in a physical space. For example, the receptacle 302 may correspond to a cart, a basket, a bag, the hands of the user, and the like. As illustrated, there are a number of items 210 in the receptacle 302. Specifically, the receptacle 302 contains carrots (indicated by item 210A), a can of soup (indicated by item 210B), a can of sauce (indicated by item 210C), a box of cereal (indicated by item 210D), and a container of cookies (indicated by item 210E).

As illustrated, a projector device 305 (which may correspond to the projector device 205 of FIG. 2) is projecting visualizations onto the items 210 in the receptacle 302 (as indicated by lines 315). As discussed above, the projector device 305 may generally correspond to or comprise any device capable of projecting visual imagery in an environment.

In some embodiments, the visualizations act as a set of notifications (e.g., notifications 125 of FIG. 1). For example, the computer vision system may project the visualizations in response to determining that the predicted caloric intake of the user meets or exceeds a threshold (e.g., a target value), or in response to a user request.

In the illustrated example, the visualizations comprise a different highlight, indication, or other visualization for each item 210 based on the nutritional content of each item 210. For example, the visualizations may be used to indicate the calorie density of each item, the total caloric value of each item, a ratio or measure based on the caloric value and other nutritional value (e.g., where higher caloric value is correlated with a higher score, and lower nutritional value is correlated with a lower score), and the like. For example, as illustrated, various amounts of boldness or emphasis (e.g., outlines of various thicknesses) are projected onto each item 210 based on the calorie density of the items.

Specifically, the item 210E (e.g., the cookies) has the highest density and/or lowest nutritional value, and therefore has the largest or most visually-intense outline, followed by the item 210D (the cereal), the item 210C (the sauce), the item 210B (the soup), and finally, the item 210A (the carrots). In this way, based on the visualizations, the user can readily perceive which item(s) contribute most towards their predicted caloric intake.

In some embodiments, to project the visualizations, the computer vision system may determine, based on the image data depicting the user's receptacle 302 (e.g., image data 115 of FIG. 1), the location(s) of one or more item(s) 210 in the receptacle 302, as discussed above. For example, based on object detection and recognition models, the computer vision system may determine the physical location of each item 210 relative to the cart. This may allow the computer vision system to project the visualizations onto each of the physical locations of the items in the receptacle 302.

Although depicted as an outline of varying size, the visualizations may take a variety of forms depending on the particular implementation. For example, in some aspects, the computer vision system may circle or outline the item(s) using different colors or line types (e.g., solid lines, dotted lines, and the like) to indicate the various nutritional information. As another example, the computer vision system may project a color overlay onto the relevant item(s), such as to depict a heat map (e.g., where highly caloric items are highlighted in red, and low-calorie options are highlighted in green).

In some embodiments, the computer vision system generates the visualizations in response to a user request. For example, the user may use an application on their phone to request that the computer vision system identify and indicate the relative caloric density or values of the item(s) 210 in their receptacle. In some embodiments, the computer vision system identifies the items having the highest calorie density. In some embodiments, the computer vision system may also consider other information, such as other nutritional value of the items (e.g., where some items may be highly caloric but also high in other nutrition, while other items may be highly caloric without substantial other nutritional value). In some such embodiments, the computer vision system may indicate item(s) that are high calorie with low other nutritional value (e.g., below a threshold).

In some embodiments, the computer vision system generates the visualizations automatically in response to determining that the predicted intake exceeds the user's target caloric intake. For example, suppose the addition of the item 210C to the receptacle 302 causes the predicted caloric intake to exceed the target. In some embodiments, the computer vision system may therefore evaluate each item 210 in the receptacle 302 to determine the calorie values and/or calorie densities of each, and/or the lowest nutritional value of each. These visualizations may then be generated to indicate the relative nutritional values of each item in the receptacle 302.

### Example Method for Generating Computer Vision-Based Notifications

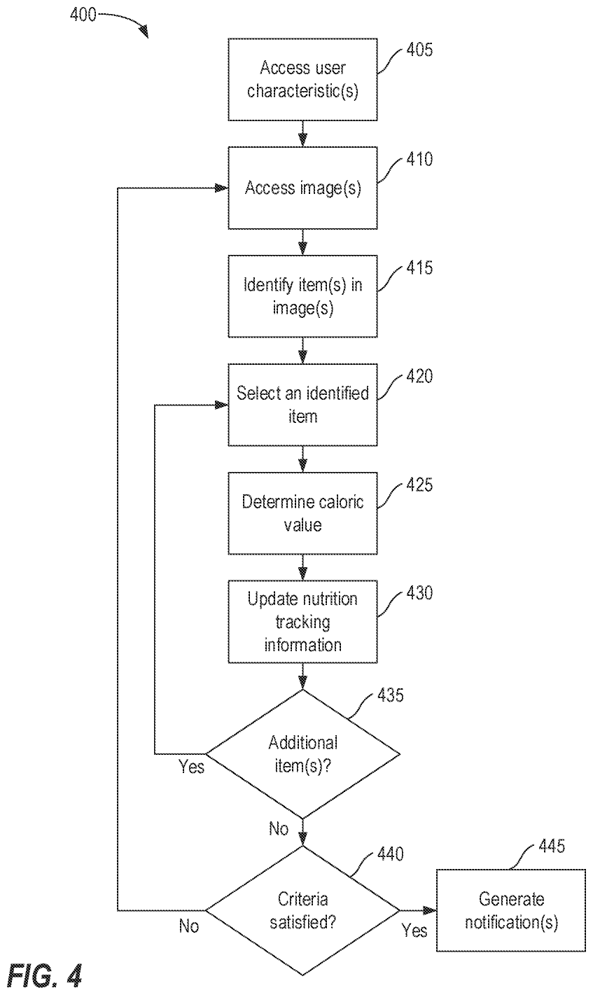

FIG. 4 is a flow diagram depicting an example method 400 for generating computer vision-based notifications, according to some embodiments of the present disclosure. In some embodiments, the method 400 is performed by a computer vision system, such as the computer vision system 105 of FIG. 1.

At block 405, the computer vision system accesses a set of user characteristics (e.g., the user characteristics 122 of FIG. 1) for a given user. As discussed above, the user characteristics may generally include a variety of contextual data, such as attributes of the given user and/or other users associated with the given user (e.g., other individuals for whom the given user is also shopping). For example, in some aspects, the user characteristics may indicate demographic information such as the age(s), gender(s), height(s), weight(s), activity level(s), caloric target(s), and the like for the given user and/or for each associated user. In some embodiments, the user characteristics may similarly include information such as the duration of time (e.g., a number of hours, days, or weeks) for which the user is shopping (e.g., how long the user expects the selected items to last before they collect more items), and/or a number of meals and/or snacks that the user plans to prepare using the selected items. As another example, in some embodiments, the user characteristics may include a predicted caloric waste for the user (or for items selected by the user, if the waste is a predefined or user-agnostic value).

At block 410, the computer vision system accesses a set of one or more images (e.g., image data 115 of FIG. 1) that depict a receptacle of the given user. For example, as discussed above, one or more imaging sensors (e.g., imaging sensors 110 of FIG. 1) such as cameras may capture image(s) of the environment, and various techniques such as facial recognition, detection of the user's smartphone or other device, and the like may be used to identify which image(s) depict the user and/or their receptacle.

At block 415, the computer vision system identifies a set of item(s) depicted in the accessed image(s). That is, the computer vision system identifies which item(s) are present in the receptacle of the given user, as depicted in the image(s). For example, in some aspects, the computer vision system processes the image(s) using one or more machine learning models trained to perform object detection and/or recognition, text recognition, and the like. As one example, an image may be processed using a first machine learning model to detect and categorize the depicted items (e.g., to indicate that a first region or set of pixels depict bananas, a second depict a can, and the like). In some embodiments, one or more of these regions may then be processed using a second model to classify the specific items depicted (e.g., to identify which brand and/or type of can is depicted).

At block 420, the computer vision system selects one of the identified items that are present in the user's receptacle. Generally, this selection may be performed using a variety of criteria or techniques (including randomly or pseudo-randomly), as each item will be evaluated during execution of the method 400. In some aspects, the computer vision system selects from the entire list of items in the user's receptacle. That is, each time the method 400 is performed, all items may be evaluated. In some embodiments, the computer vision system selects from a subset of the items in the receptacle. For example, the computer vision system may determine which item(s) are newly added (e.g., which were added since the last time the method 400 was performed), and may select an item from this subset of new items. This can reduce computational expense of the method 400.

At block 425, the computer vision system determines a caloric value of the selected item. For example, as discussed above, the computer vision system may use one or more item mappings (e.g., item mappings 120 of FIG. 1) to identify the total caloric value of the item, the per-serving caloric value of the item, the caloric density of the item, and/or other nutritional value of the item.

At block 430, the computer vision system updates nutrition tracking information for the user. For example, as discussed above, the computer vision system may use the nutrition tracking information to track the total caloric value (or other nutritional value) of the items in the user's receptacle during the current shopping trip.

At block 435, the computer vision system determines whether there is at least one additional item, in the user's receptacle, remaining to be processed. If so, the method 400 returns to block 420. If not, the method 400 continues to block 440. Although the illustrated example depicts an iterative process (e.g., selecting and evaluating each item in sequence) for conceptual clarity, in some aspects, the computer vision system may select and/or process multiple detected items in parallel.

At block 440, the computer vision system determines whether one or more criteria are satisfied (based on the updated nutrition tracking information). For example, in some embodiments, the computer vision system may determine whether the total caloric value of the nutrition tracking information exceeds a defined maximum or target. In some embodiments, as discussed above, the computer vision system may evaluate the criteria in the context of the user characteristics.

For example, the computer vision system may subtract an estimated caloric waste from the total value (or from each item, if the waste is item-specific), divide the total caloric value by the number of individuals for whom the user is shopping, and/or divide the total caloric value by the number of meals or days for which the user is shopping. In this way, the computer vision system can generate an accurate prediction regarding the caloric intake of the user (e.g., the number of calories per day and/or per meal). In some embodiments, for example, the computer vision system may determine whether the predicted caloric intake (e.g., per day) exceeds a user-defined caloric target.

If, at block 440, the computer vision system determines that the criteria are not met (e.g., the predicted caloric intake of the given user is below their target), the method 400 returns to block 410 to continue evaluating the transaction/receptacle. If, at block 440, the computer vision system determines that the criteria are met, the method 400 continues to block 445. At block 445, the computer vision system generates and outputs one or more notifications (e.g., notifications 125 of FIG. 1), as discussed above.

For example, in some embodiments, the computer vision system may generate a textual alert or notification and transmit this notification to the user's device (e.g., via text message or in-app alert). In some embodiments, the notification indicates which item(s) have high calorie density and/or caloric value, which items have low nutritional value, and the like. In some embodiments, the notification allows the user to sort and/or filter the items based on a variety of criteria, such as to filter out certain items, to sort by calorie density or caloric value, and the like.

As another example, in some embodiments, the computer vision system may generate and project one or more visualizations onto the item(s) in the user's receptacle. For example, the computer vision system may project a first visualization (e.g., an “X”) over items that do not meet certain criteria (e.g., that have a high calorie density with a lower nutritional value). As another example, the computer vision system may project visualizations indicating (e.g., highlighting or outlining) the N items that are most calorie-dense, and/or indicating the relative caloric values or densities of these items (e.g., using a heat map approach).

In these ways, the computer vision system can allow the user to quickly identify which item(s) are problematic for their goals, and eliminate these items from the selection.

### Example Method for Outputting Computer Vision-Based Notifications

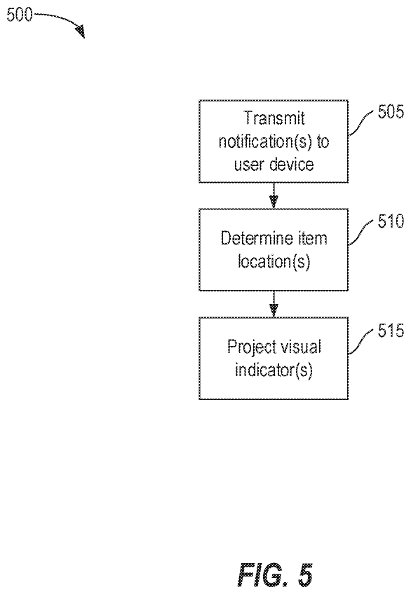

FIG. 5 is a flow diagram depicting an example method 500 for outputting computer vision-based notifications, according to some embodiments of the present disclosure. In some embodiments, the method 500 is performed by a computer vision system, such as the computer vision system 105 of FIG. 1. In some embodiments, the method 500 provides additional detail for block 445 of FIG. 4.

At block 505, the computer vision system transmits one or more notification(s) to a user device associated with the given user for whom the computer vision system is tracking nutritional information. For example, as discussed above, the computer vision system may generate and transmit text message(s), application alerts, and the like. In some embodiments, the computer vision system transmits the notifications via one or more wireless networks. For example, the computer vision system may transmit the notifications via a local network (e.g. a Bluetooth connection, or a wireless local area network (WLAN) such as a WiFi network provided in the space). In some embodiments, the computer vision system may transmit the notifications via one or more broader networks, such as via the Internet, via a cellular network, and the like.

In some embodiments, the notifications indicate that the user is predicted to exceed their caloric goals, based on the item(s) in the receptacle. In some embodiments, the notifications can specifically indicate a subset of the items having a high calorie value, a high calorie density, and/or a low nutritional value. For example, the notification may indicate the N items having the highest caloric value, and/or may indicate items in the top N % of caloric value (e.g., the top 20% of items in the receptacle). In some embodiments, the notification may indicate the caloric value, caloric density, and/or other nutritional value for all items in the receptacle.

At block 510, the computer vision system determines the location of each item in the receptacle. For example, based on evaluating the image(s) depicting the receptacle, the computer vision system may identify which pixel(s) correspond to each item, as well as the location of each item relative to the receptacle (e.g., relative to the pixels that correspond to the perimeter of the receptacle). For example, the computer vision system may determine that a cereal item is located in the front-left corner of the user's cart, while a tomato item is located in the center of the cart.

In some embodiments, the computer vision system identifies the locations of each item. In some embodiments, the computer vision system determines to locations of a subset of the items. For example, the computer vision system may identify the location of one or more items having the highest caloric values and/or caloric densities, and/or having the lowest other nutritional value.

At block 515, the computer vision system projects one or more visual indicators (e.g., visualization 220 of FIG. 2) onto the determined item locations in the physical space. For example, as discussed above, the computer vision system may project a symbol (e.g., an “X”) indicating that a given item should not be selected (e.g., because its caloric value is too high to justify its nutritional value, in view of the specific user's target goals). As another example, the computer vision system may project indicators such as coloring, outlines, or other visual effects indicating the relative caloric values, densities, and/or nutritional values of the items.

### Example Method for Generating Notifications

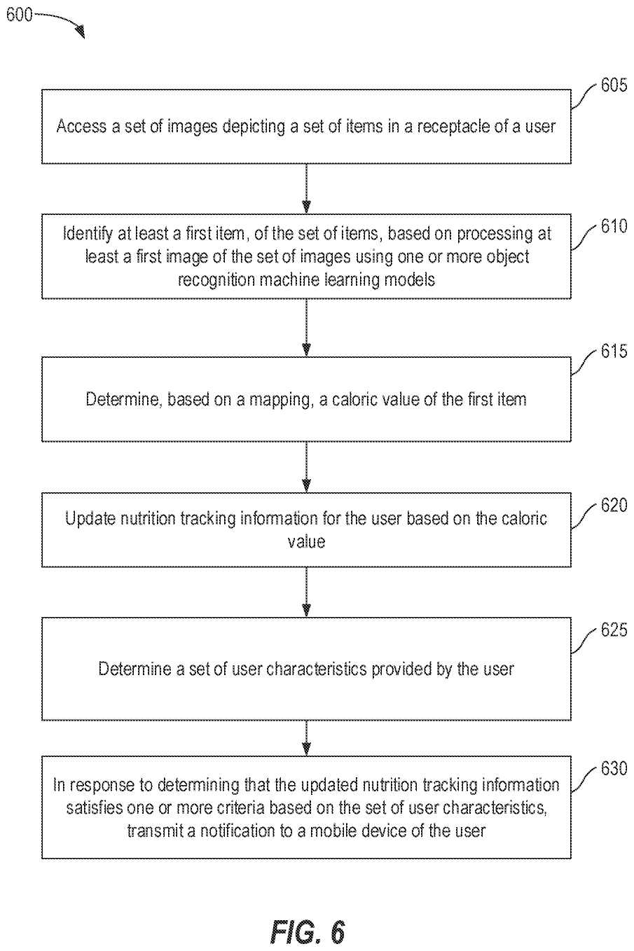

FIG. 6 is a flow diagram depicting an example method 600 for generating notifications, according to some embodiments of the present disclosure. In some embodiments, the method 600 is performed by a computer vision system, such as the computer vision system 105 of FIG. 1.

At block 605, a set of images (e.g., image data 115 of FIG. 1) depicting a set of items (e.g., items 130 of FIG. 1 and/or items 210 of FIGS. 2-3) in a receptacle (e.g., receptacle 202 of FIG. 2 and/or receptacle 302 of FIG. 3) of a user (e.g., user 135 of FIG. 1) is accessed.

At block 610, at least a first item, of the set of items, is identified based on processing at least a first image of the set of images using one or more object recognition machine learning models.

At block 615, based on a mapping (e.g., item mappings 120 of FIG. 1), a caloric value of the first item is determined.

At block 620, nutrition tracking information for the user is updated based on the caloric value.

At block 625, a set of user characteristics (e.g., user characteristics 122 of FIG. 1) provided by the user is determined.

At block 630, in response to determining that the updated nutrition tracking information satisfies one or more criteria based on the set of user characteristics, a notification (e.g., notification 125 of FIG. 1) is transmitted to a mobile device of the user.

### Example Computing Device for Computer Vision-Based Nutrition Tracking

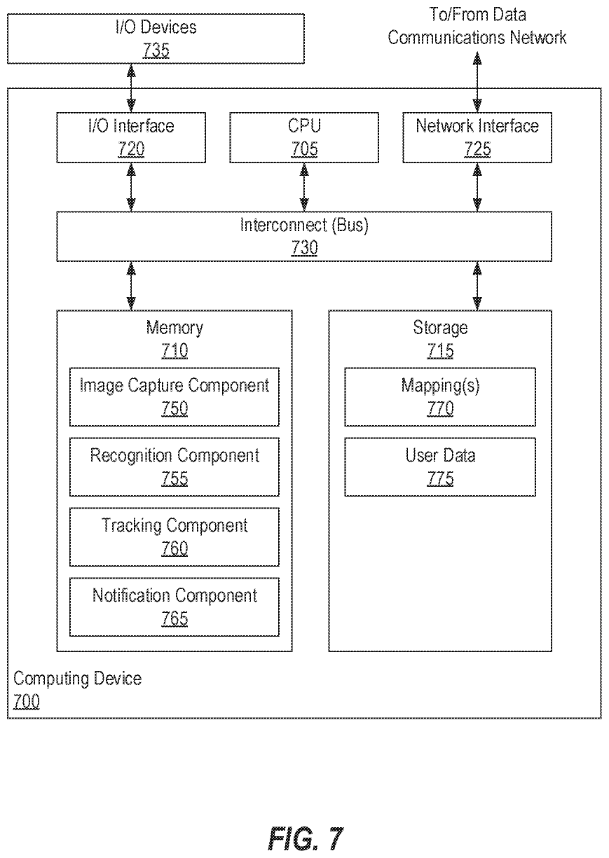

FIG. 7 depicts an example computing device 700 for computer vision-based nutrition tracking, according to some embodiments of the present disclosure.

Although depicted as a physical device, in some embodiments, the computing device 700 may be implemented using virtual device(s), and/or across a number of devices (e.g., in a cloud environment). In some embodiments, the computing device 700 corresponds to or comprises a computer vision system, such as the computer vision system 105 of FIG. 1.

As illustrated, the computing device 700 includes a CPU 705, memory 710, storage 715, one or more network interfaces 725, and one or more I/O interfaces 720. In the illustrated embodiment, the CPU 705 retrieves and executes programming instructions stored in memory 710, as well as stores and retrieves application data residing in storage 715. The CPU 705 is generally representative of a single CPU and/or GPU, multiple CPUs and/or GPUs, a single CPU and/or GPU having multiple processing cores, and the like. The memory 710 is generally considered to be representative of a random access memory. Storage 715 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 735 (such as keyboards, monitors, etc.) are connected via the I/O interface(s) 720. Further, via the network interface 725, the computing device 700 can be communicatively coupled with one or more other devices and components (e.g., via a network, which may include the Internet, local network(s), and the like). As illustrated, the CPU 705, memory 710, storage 715, network interface(s) 725, and I/O interface(s) 720 are communicatively coupled by one or more buses 730. In the illustrated embodiment, the memory 710 includes an image capture component 750, a recognition component 755, a tracking component 760, and a notification component 765.

Although depicted as discrete components for conceptual clarity, in some embodiments, the operations of the depicted components (and others not illustrated) may be combined or distributed across any number of components. Further, although depicted as software residing in memory 710, in some embodiments, the operations of the depicted components (and others not illustrated) may be implemented using hardware, software, or a combination of hardware and software.

In the illustrated embodiment, the image capture component 750 (which may correspond to and/or use the imaging sensors 110 of FIG. 1) is configured to capture image data (e.g., image data 115 of FIG. 1) depicting items (e.g., items 130 of FIG. 1, and/or items 210 of FIGS. 2-3) being selected by users (e.g., users 135 of FIG. 1) in a physical environment (e.g., items in the user's receptacle 202 of FIG. 2 and/or receptacle 302 of FIG. 3). For example, the image capture component 750 may use a variety of cameras or other sensors arranged in various locations throughout a physical space to capture image data of the items and receptacles. In some aspects, the image capture component 750 continuously captures image data (e.g., capturing video data, or capturing image frames every N seconds). In some embodiments, the image capture component 750 captures image data upon request (e.g., when a user requests that their selections be evaluated).

In the illustrated example, the recognition component 755 generally uses one or more algorithms, models, and/or operations to evaluate image data (e.g., captured by the image capture component 750) in order to identify items depicted in the image data. For example, as discussed above, the image capture component 750 may use one or more machine learning models (e.g., CNNs), such as using an object detection and/or segmentation model to identify items in the receptacle, item recognition and/or text recognition models to identify the specific brands, types, or other identifier for the items, and the like.

In the illustrated embodiment, the tracking component 760 may be configured to generate and update a list of items (e.g., in a nutrition tracking information dataset) based on the items detected by the recognition component 755. For example, for each user (or for each user who opted in to the system), the tracking component 760 may update nutrition information for the user as the items are recognized in their receptacle. In some embodiments, the tracking component 760 can determine or predict the total caloric intake of the user, such as by evaluating item mappings (e.g., item mappings 120 of FIG. 1) to determine a total caloric value of the items. Similarly, in some embodiments, the tracking component 760 can evaluate contextual information such as estimated caloric waste(s), a number and/or attributes of other individuals for whom the user is shopping, a number of meals and/or duration of time for which the user is shopping, and the like. This contextual information can substantially improve the accuracy of the predicted caloric intake.

In the illustrated example, the notification component 765 may be configured to generate and/or provide notifications to users, based on the nutrition tracking information provided by the tracking component 760. For example, as discussed above, the notification component 765 may generate textual or other notifications and transmit them to the mobile device of the user. In some embodiments, as discussed above, the notification component 765 may project visualizations onto the items in the user's receptacle, allowing the user to quickly evaluate the nutritional and/or caloric values of each item.

In the illustrated example, the storage 715 may include a set of mappings 770 (which may correspond to the item mappings 120 of FIG. 1) and a set of user data 775 (which may correspond to the user characteristics 122 of FIG. 1). In some embodiments, the mappings 770 may include nutritional information (e.g., caloric value, vitamin amounts, carbohydrate amounts, and the like) for the items that can be selected in the physical location. In some embodiments, the user data 775 may include various user characteristics or attributes, such as the demographics of the user, the number of meals and/or individuals the user is shopping for, and the like. In some embodiments, the aforementioned data may be saved in a remote database that connects to the computing device 700 via a network.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the following, reference is made to embodiments presented in this disclosure. However, the scope of the present disclosure is not limited to specific described embodiments. Instead, any combination of the following features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.”

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications (e.g., the computer vision system) or related data available in the cloud. For example, the computer vision system could execute on a computing system in the cloud and evaluate image data to track nutrition information. In such a case, the computer vision system could predict caloric intake for users, and store the tracking information and/or predictions at a storage location in the cloud. Doing so allows a user to access this information from any computing system attached to a network connected to the cloud (e.g., the Internet).

While the foregoing is directed to embodiments of the present invention, other and further embodiments of the invention may be devised without departing from the basic scope thereof, and the scope thereof is determined by the claims that follow.

What is claimed is:

1. A method, comprising:
accessing a set of images depicting a set of items in a receptacle of a user;
identifying at least a first item, of the set of items, based on processing at least a first image of the set of images using one or more object recognition machine learning models;
determining, based on a mapping, a caloric value of the first item;
updating nutrition tracking information for the user based on the caloric value;
determining a set of user characteristics provided by the user; and
in response to determining that the updated nutrition tracking information satisfies one or more criteria based on the set of user characteristics, transmitting a notification to a mobile device of the user.

2. The method of claim 1, wherein the set of user characteristics comprise at least one of:
(i) a number of days or meals for which the user is shopping,
(ii) a number of individuals for whom the user is shopping, or
(iii) a target caloric intake for the user.

3. The method of claim 1, wherein updating the nutrition tracking information comprises predicting a caloric intake for the user based at least in part on the caloric value of the first item.

4. The method of claim 3, wherein predicting the caloric intake for the user comprises:
determining an estimated caloric waste for the first item; and
subtracting the estimated caloric waste from the caloric value.

5. The method of claim 3, wherein the notification indicates that the user is predicted to exceed a target caloric intake if the first item is retained by the user.

6. The method of claim 1, further comprising, in response to determining that the updated nutrition tracking information satisfies one or more criteria:
determining, based on the set of images, a location of the first item in the receptacle of the user; and
projecting, via one or more projection devices, a visual indication onto the first item at the location.

7. The method of claim 1, further comprising:
receiving, via the mobile device of the user, a request to evaluate items in the receptacle of the user;
identifying each item of the set of items based on processing the set of images using the one or more object recognition machine learning models;
determining, based on the mapping, a respective caloric value of each respective item in the set of items;
determining a subset of items, from the set of items, having caloric values satisfying one or more caloric criteria; and
projecting, via one or more projection devices, a visual indication onto each item of the subset of items.

8. A system comprising:
one or more memories collectively storing computer-executable instructions; and
one or more processors configured to collectively execute the computer-executable instructions and cause the system to perform an operation, comprising:
accessing a set of images depicting a set of items in a receptacle of a user;
identifying at least a first item, of the set of items, based on processing at least a first image of the set of images using one or more object recognition machine learning models;
determining, based on a mapping, a caloric value of the first item;
updating nutrition tracking information for the user based on the caloric value;
determining a set of user characteristics provided by the user; and
in response to determining that the updated nutrition tracking information satisfies one or more criteria based on the set of user characteristics, transmitting a notification to a mobile device of the user.

9. The system of claim 8, wherein the set of user characteristics comprise at least one of:
(i) a number of days or meals for which the user is shopping,
(ii) a number of individuals for whom the user is shopping, or
(iii) a target caloric intake for the user.

10. The system of claim 8, wherein updating the nutrition tracking information comprises predicting a caloric intake for the user based at least in part on the caloric value of the first item.

11. The system of claim 10, wherein predicting the caloric intake for the user comprises:
determining an estimated caloric waste for the first item; and
subtracting the estimated caloric waste from the caloric value.

12. The system of claim 10, wherein the notification indicates that the user is predicted to exceed a target caloric intake if the first item is retained by the user.

13. The system of claim 8, the operation further comprising, in response to determining that the updated nutrition tracking information satisfies one or more criteria:
determining, based on the set of images, a location of the first item in the receptacle of the user; and
projecting, via one or more projection devices, a visual indication onto the first item at the location.

14. The system of claim 8, the operation further comprising:
receiving, via the mobile device of the user, a request to evaluate items in the receptacle of the user;
identifying each item of the set of items based on processing the set of images using the one or more object recognition machine learning models;
determining, based on the mapping, a respective caloric value of each respective item in the set of items;
determining a subset of items, from the set of items, having caloric values satisfying one or more caloric criteria; and
projecting, via one or more projection devices, a visual indication onto each item of the subset of items.

15. A computer program product comprising one or more computer-readable storage media having computer-readable program code collectively embodied therewith, the computer-readable program code collectively executable by one or more computer processors to perform an operation comprising:
accessing a set of images depicting a set of items in a receptacle of a user;
identifying at least a first item, of the set of items, based on processing at least a first image of the set of images using one or more object recognition machine learning models;
determining, based on a mapping, a caloric value of the first item;
updating nutrition tracking information for the user based on the caloric value;
determining a set of user characteristics provided by the user; and
in response to determining that the updated nutrition tracking information satisfies one or more criteria based on the set of user characteristics, transmitting a notification to a mobile device of the user.

16. The computer program product of claim 15, wherein the set of user characteristics comprise at least one of:
(i) a number of days or meals for which the user is shopping,
(ii) a number of individuals for whom the user is shopping, or
(iii) a target caloric intake for the user.

17. The computer program product of claim 15, wherein updating the nutrition tracking information comprises predicting a caloric intake for the user based at least in part on the caloric value of the first item.

18. The computer program product of claim 17, wherein predicting the caloric intake for the user comprises:
determining an estimated caloric waste for the first item; and
subtracting the estimated caloric waste from the caloric value.

19. The computer program product of claim 15, the operation further comprising, in response to determining that the updated nutrition tracking information satisfies one or more criteria:
determining, based on the set of images, a location of the first item in the receptacle of the user; and
projecting, via one or more projection devices, a visual indication onto the first item at the location.

20. The computer program product of claim 15, the operation further comprising:
receiving, via the mobile device of the user, a request to evaluate items in the receptacle of the user;
identifying each item of the set of items based on processing the set of images using the one or more object recognition machine learning models;
determining, based on the mapping, a respective caloric value of each respective item in the set of items;
determining a subset of items, from the set of items, having caloric values satisfying one or more caloric criteria; and
projecting, via one or more projection devices, a visual indication onto each item of the subset of items.
